(19) United States
(12) Patent Application Publication
(10) Pub. No.: US 2024/0082001 A1
(43) Pub. Date: Mar. 14, 2024

(54) APPARATUS AND METHOD FOR TREATING NATIVE VALVE LEAFLETS IN HUMAN HEART

(71) Applicant: Sinomed Cardiovita Technology Inc., Suzhou (CN)

(72) Inventors: Jianhua Sun, Beijing (CN); Tianzhu Li, Tianjin (CN); Jianxiang Ma, Tianjin (CN); Jinhong Zhao, Tianjin (CN); Lei Meng, Tianjin (CN)

(73) Assignee: Sinomed Cardiovita Technology Inc., Suzhou (CN)

(21) Appl. No.: 18/047,515

(22) Filed: Oct. 18, 2022

(30) Foreign Application Priority Data
Sep. 8, 2022 (CN) 202211096015.3

(51) Int. Cl. A61F 2/24

(52) U.S. Cl. CPC A61F 2/2466; A61F 2/2445; A61F 2/2454; A61F 2220/0016; A61F 2220/0025

(57) ABSTRACT

A native leaflet treatment system and method for treating native heart valve leaflets before heart valve repair or replacement procedure. The treatment system includes a delivery apparatus configured to enter the heart, and a native leaflet treatment device stored in the delivery apparatus in a compressed state. The treatment device includes a clamping structure, which is configured to retain at least a portion of the native leaflets in the deployed state. The treatment device is delivered into the heart using the delivery apparatus, and released near the native heart valve, where the treatment device expands and the clamping structure captures and holds the native leaflet, such that the native leaflets will no longer block blood flow. This improves the dynamics of the blood flow which facilitates subsequent surgical procedures (minimally-invasive surgery or open surgery) for heart valve repair or replacement.

### BACKGROUND OF THE INVENTION

### Field of the Invention

This invention relates to medical devices and methods, and in particular, it relates to apparatus and methods for treating native valve leaflets in a heart prior to heart valve repair or replacement procedures.

### Description of Related Art

The human heart has four chambers and four valves. The heart valves control the directions of blood flow. Fully-functional heart valves ensure that proper blood circulation is maintained during the cardiac cycle. When heart valve regurgitation, or leakage, occurs due to primary or secondary diseases and the valves cannot maintain normal heart functions, surgical intervention may be needed to treat the heart valves and restore normal heart function.

Currently, standard heart valve treatment options include surgical intervention to repair or replace the heart valve, such as by open surgery or minimally-invasive surgery (e.g. endovascularly or transcatheterly). Regardless of the types of procedures, the native leaflets of the heart valve should be appropriately treated prior to the valve repair or replacement, e.g., to manipulate the leaflet positions or trim the leaflet shapes, so that the native leaflets will no longer block blood flow. This improves the dynamics of the blood flow which facilitates subsequent surgical procedures for heart valve repair or replacement.

Therefore, there is a need for a device for treating the native leaflets of the heart valve prior to valve repair or replacement surgery.

### SUMMARY

Accordingly, the present invention is directed to a native valve leaflet treatment apparatus and method that substantially obviates one or more of the problems due to limitations and disadvantages of the related art.

An object of the present invention is to provide a native valve leaflet treatment system and related method for treating the native valve leaflets before heart valve repair or replacement procedures, such as folding or clipping the native leaflet.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a native leaflet treatment system for treating native leaflets of a native valve of a heart, which includes: a delivery apparatus, including a sheath and a tip, configured to be inserted into the heart; and a native leaflet treatment device, having a compressed state for delivery and a deployed state, wherein in the compressed state, the treatment device is compressed and stored inside the delivery apparatus, and in the deployed state, the treatment device is expanded, wherein the treatment device includes a clamping structure, and in the deployed state, the clamping structure is configured to capture and retain at least a portion of the native leaflets.

In some embodiments, the treatment device further includes: an anchoring structure; and a plurality of connecting bridges connecting the clamping structure and the anchoring structure, wherein the anchoring structure is connected at first ends of the connecting bridges, and wherein in the deployed state, the anchoring structure is configured to abut an annulus of the native valve.

In some embodiments, the treatment device further includes a plurality of piercing elements, connected to the anchoring structure and having sharp tips pointing toward the clamping structure, the sharp tips being configured to pierce into the annulus of the native valve in the deployed state.

In some embodiments, the clamping structure, the anchoring structure and the connecting bridges are formed of a shape memory material.

In some embodiments, the treatment device further includes a plurality of proximal locking members joined to the anchoring structure and configured to be detachably coupled to the sheath of the delivery apparatus when the treatment device is stored in the delivery apparatus.

In some embodiments, the treatment device further includes a plurality of distal locking members joined to the clamping structure and configured to be detachably coupled to the tip of the delivery apparatus when the treatment device is stored in the delivery apparatus.

In some embodiments, the plurality of proximal locking members are respectively joined to upper points of the anchoring structure, and the plurality of distal locking members are respectively joined to lower points of the clamping structure.

In some embodiments, wherein the plurality of proximal locking members are respectively joined to upper points of the anchoring structure, wherein the treatment device further includes a plurality of legs, wherein each leg is joined at its upper end to an upper point of the clamping structure and bends downwardly in the deployed state, and wherein the plurality of distal locking members are respectively joined to lower ends of the plurality of legs.

In some embodiments, each of the clamping structure and the anchoring structure is formed of connected struts, and wherein in the deployed state, each of the clamping structure and the anchoring structure forms an undulating structure, each connecting bridge is connected to a lower point of the undulating structure of the anchoring structure and a lower point of the undulating structure of the clamping structure, and both the clamping structure and the anchoring structure extend upwardly and outwardly from the respective lower points where they are joined to the connecting bridges.

In some embodiments, in the deployed state, the anchoring structure forms an angle between 0 and 75 degrees with respect to a horizontal plane of the treatment device.

In some embodiments, treatment device is formed of a plurality of repeating units connected to each other and arranged in an angular direction around an longitudinal axis of the treatment device.

In some embodiments, in a top view in the deployed state, the treatment device has an open shape forming an incomplete ring around the longitudinal axis.

In some embodiments, in a top view in the deployed state, the treatment device has a closed shape forming a complete ring around the longitudinal axis.

In some embodiments, the delivery apparatus further includes a wire which passes through the sheath and is connected at its distal end to the tip of the delivery apparatus.

In another aspect, the present invention provides a native leaflet treatment method for treating the native leaflets using a native leaflet treatment system, the treatment system including a delivery apparatus and a native leaflet treatment device stored inside the delivery apparatus in a compressed state, the native leaflet treatment device including a clamping structure, and the native leaflet treatment device being configured to self-expand to a deployed state upon release from the delivery apparatus, the method including: inserting the delivery apparatus with the treatment device stored therein into an atrium of the heart and through the native valve; releasing the treatment device from the delivery apparatus, wherein the treatment device self-expands to the deployed state wherein the clamping structure captures and retains at least a portion of the native leaflets; and withdrawing the delivery apparatus from the heart.

In some embodiments, the delivery apparatus including a sheath and a tip located a s distal end of the sheath, and wherein the releasing step includes: releasing a proximal portion of the treatment device from the sheath while retaining a distal end of the treatment device in the tip, wherein the released proximal portion of the treatment device self-expands to abut an annulus of the native valve; and thereafter, releasing the distal end of the treatment device from the tip.

In some embodiments, the delivery apparatus including a sheath and a tip located a s distal end of the sheath, and wherein the releasing step includes: releasing a distal portion of the treatment device including the clamping structure from the tip and the sheath while retaining a proximal end of the treatment device in the sheath, wherein the clamping structure self-expands to capture and retain at least a portion of the native leaflets; and thereafter, releasing the proximal end of the treatment device from the sheath, wherein the treatment device self-expands further to abut an annulus of the native valve.

In some embodiments, the treatment device in the deployed state is anchored to an annulus of the native valve by a plurality of piercing elements.

In another aspect, the present invention provides a native leaflet treatment method of treating native leaflets of a native valve of a heart, including delivering a treatment device into the heart transcatheterly, wherein the treatment device self-expands to capture and retain the native leaflets, before repairing or replacing the native valve.

In some embodiments, the atrium is the left atrium and the native valve is a mitral valve.

It is to be understood that both the foregoing general description and the following detailed description are exemplary and explanatory and are intended to provide further explanation of the invention as claimed.

### BRIEF DESCRIPTION OF THE DRAWINGS

Drawings are provided here to more clearly describe embodiments of the present invention. These drawings illustrate some embodiments of the present invention. Those of ordinary skill in the art may derive other drawings based on the drawings provided here without creative work.

FIG. 1 is a perspective view illustrating a native heart valve leaflet treatment device according to an embodiment of the present invention.

FIG. 1A is a top view illustrating the native leaflet treatment device of FIG. 1.

FIG. 2 is another perspective view illustrating the native leaflet treatment device of FIG. 1.

FIG. 3 illustrates a delivery apparatus containing the native leaflet treatment device being delivered into the heart according to an embodiment of the present invention.

FIGS. 4 and 5 illustrate two steps of deploying the treatment device of FIG. 1 into the heart to treat the native leaflets according to an embodiment of the present invention.

FIG. 6 is a perspective view illustrating a native heart valve leaflet treatment device according to another embodiment of the present invention.

FIGS. 7 and 8 illustrate two steps of deploying the treatment device of FIG. 6 into the heart to treat the native leaflet.

FIG. 9 is a perspective view illustrating a native heart valve leaflet treatment device according to yet another embodiment of the present invention.

FIGS. 10 and 11 illustrate two steps of deploying the treatment device of FIG. 9 into the heart to treat the native leaflet.

FIG. 12 illustrates an alternative deployment method of native leaflet treatment using the treatment device of FIG. 1.

Reference symbols used in the drawings: 1. clamping structure; 2. anchoring structure; 3. connecting bridge; 4. piercing element; 5. proximal locking member; 6. distal locking member; 7. right atrium; 8. left atrium; 9. left ventricle; 10. delivery apparatus; 11. tip of delivery apparatus; 12. connecting wire; 13. native leaflet; 14. native leaflet treatment device; 15. alternative native leaflet treatment device; 16. alternative native leaflet treatment device; 17. leg of clamping structure; 18. distal locking member.

### DETAILED DESCRIPTION OF PREFERRED EMBODIMENTS

Embodiments of the present invention are described below with reference to the drawings. These embodiments are only some but not all possible embodiments of the present invention. Based on the descriptions here, those of ordinary skill in the art may derive other embodiments, which also fall within the scope of the protected invention.

In the descriptions below, terms such as center, up, down, left, right, vertical, horizontal, inside, outside, etc. should be understood to indicate positions or orientations in reference to what is shown in the drawings. They are used to conveniently describe the embodiments, and do not imply that all components or elements must have such specific positions or orientations or must operate in relation to such specific positions or orientations. These terms do not limit the scope of the invention.

In the descriptions below, terms such as coupling, connection, mounting, etc. should be understood broadly. Unless otherwise described, connection may be fixed connection or removeable connection, or integral connection as one piece; it can include mechanical connection or electrical connection; it can be direction connection or indirection connection via an intermediary, or can mean an internal connection of two components. The meaning of these terms can be properly understood by those of ordinary skill in the art based on context.

Further, the various embodiments of the present invention may be combined when suitable.

### First Embodiment

This embodiment of the present invention provides a treatment device and system for treating native leaflets of a heart valve, which may be suitably used to treat native leaflets of the mitral valve or tricuspid valve. Current minimally invasive valve repair or replacement procedures cannot properly treat the native anterior leaflets of the mitral valve. The native leaflets of the aortic valve and pulmonary valve typically do not need treatment before the repair or replacement procedures.

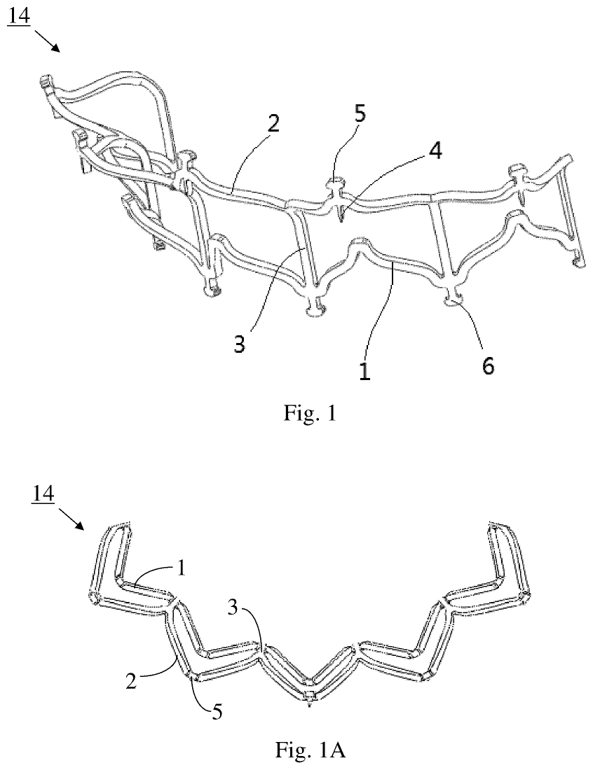

The native heart valve leaflet treatment system according to this embodiment includes a delivery apparatus 10 and a native leaflet treatment device 14. The delivery apparatus 10 includes a sheath (also represented by reference symbol 10 in the drawings) and a tip 11 located at the distal end of the sheath. The delivery apparatus 10 has a structure configured to enter the heart, with an entry profile of 50 French (F) or less. The treatment device 14 has a compressed state for delivery, in which it is compressed and stored inside the sheath 10 of the delivery apparatus, and a deployed state (FIGS. 1, 2 and 5) in which it is released from the sheath 10 and self-expanded. The treatment device 14 is formed of multiple repeating units connected to each other and arranged in an angular direction around a longitudinal axis in the deployed state.

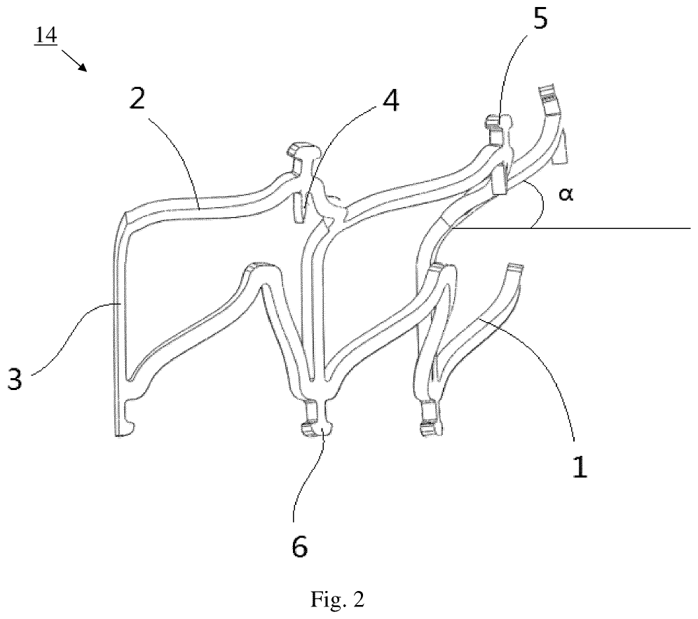

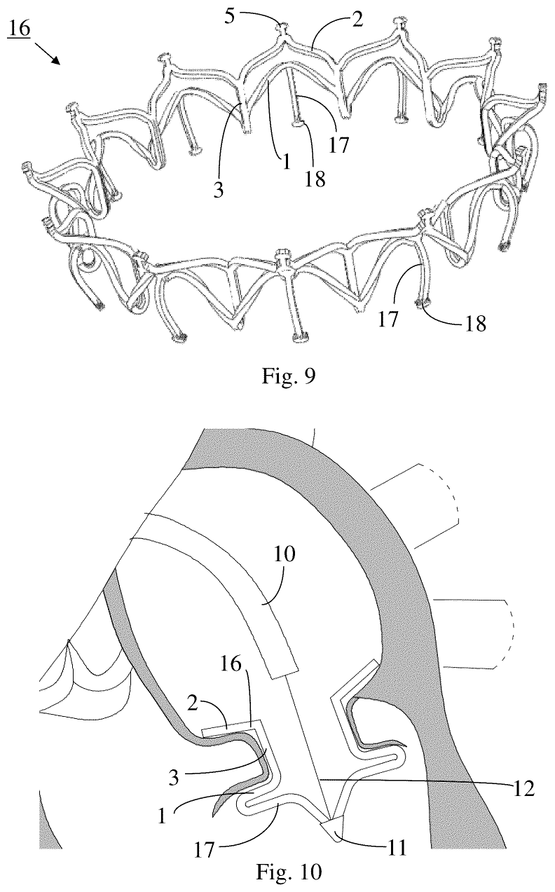

In the top view (when viewed in the longitudinal direction), the expanded treatment device 14 may have a circular shape, or a D shape (i.e. with a relative straight side and a curved side) or oval shape that matches the anatomical shape of the mitral valve, or a part thereof. In various embodiments, the device may be a closed structure which forms a complete ring in the top view (referred to as an O shape, see FIGS. 6 and 9, described in more detail later), or an open structure which forms an incomplete ring (referred to as a C shape, as shown in FIGS. 1, 1A, 2).

As shown in FIGS. 1 and 2, in the deployed state, the treatment device 14 includes a clamping structure 1 configured to clamp the native leaflets 13. The clamping structure 1 is connected to an anchoring structure 2 by connecting bridges 3. The anchoring structure 2 is disposed at a more proximal (more upstream in the direction of blood flow) position relative to the clamping structure 1, and is configured to abut the annulus of the native heart valve from the atrial side. In the illustrated embodiment, both the clamping structure 1 and anchoring structure 2 are formed of connected struts and have an undulating shape, with the connecting bridges 3 connected at the lower points of the undulating structures. In the side view (viewed in a direction perpendicular to the longitudinal direction), both the clamping structure 1 and the anchoring structure 2 extend upwardly and outwardly from the lower points where they are joined to the connecting bridges 3.

Note that in the orientation of the majority of the drawings in this disclosure, the terms “distal” and “proximal” refer to positions relative to the delivery apparatus; the term “down” refers to the direction from the atrium to the ventricle (direction of blood flow), which is also the proximal to distal direction in these examples.

As shown in FIG. 2, in the side view, the anchoring structure 2 forms an angle α with respect to the horizontal plane, which may be between 0 and 50 degrees. Alternatively, the anchoring structure 2 may have a saddle shape, with a slope that changes from a more vertical direction at the inner diameter to a more horizontal direction at the outer diameter, where the angle α changes between 0 and 75 degrees. Here, the horizontal plane may be defined as a plane passing through the upper edge or lower edge of the treatment device 14.

When the treatment device 14 is in the deployed state and situated around the native heart valve, the anchoring structure 2 is anchored to the annulus tissue, and the clamping structure 1 captures the native leaflets 13. The clamping structure 1 and the connecting bridges 3 fold the native leaflets 13 and push them outwardly and upwardly toward the anchoring structure 2, so that the native leaflets 13 no longer block blood flow. This position adjustment achieves the desired treatment of the native leaflets 13. The treatment improves the dynamics of the blood flow which facilitates subsequent surgical treatment.

To anchor the treatment device, the anchoring structure 2 includes a plurality of piercing elements 4, each having a sharp tip that points downwardly (toward the ventricle and the clamping structure 1). In the deployed state, the piercing elements 4 are configured to pierce into the annulus tissue to anchor the treatment device relative to the native heart valve. In a preferred embodiment, each piercing element 4 is a pin or rivet. In the illustrated embodiment, the piercing elements 4 are located near the outermost and uppermost points of the undulating structure of the anchoring structure 2, but they may alternatively be located at other positions along the anchoring structure 2.

Preferably, the clamping structure 1, anchoring structure 2 and connecting bridges 3 are formed of a shape memory material, such as Nitinol.

The treatment device 14 further has a number of locking structures for releasably attaching the treatment device 14 to the delivery apparatus. The locking structures include proximal locking members 5 and distal locking members 6. The proximal locking members 5 are fixedly joined to the anchoring structure 2, and each has a upwardly protruding head with a shape configured to be detachably coupled to the sheath 10 of the delivery apparatus when the treatment device 14 is stored in the sheath. In the illustrated embodiment, the proximal locking members 5 are located at the uppermost points of the undulating structure of the anchoring structure 2. The distal locking members 6 are fixedly joined to the clamping structure 1, and each has a downwardly protruding head with a shape configured to be detachably coupled to the tip 11 of the delivery apparatus when the treatment device 14 is stored in the sheath. In the illustrated embodiment, the distal locking member 6 are located at the lowermost points of the undulating structure of the clamping structure 1. The detachable coupling of the proximal locking member 5 and distal locking member 6 to the sheath 10 and tip 11 may be accomplished by snaps or locks or other suitable structures inside the delivery apparatus.

A connecting wire 12 is attached to the tip 11 of the delivery apparatus, and allows the tip to be separated from the sheath in a controlled manner. The wire 12 is made of a relatively rigid material, such as a metal wire. The wire 12 passes through the sheath 10 to connect to a control mechanism at the other end of the delivery system located outside of the patient's body.

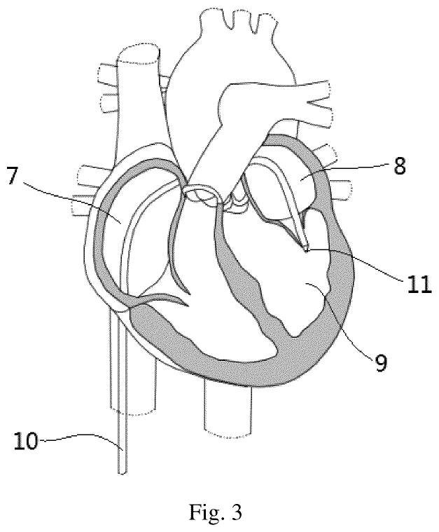

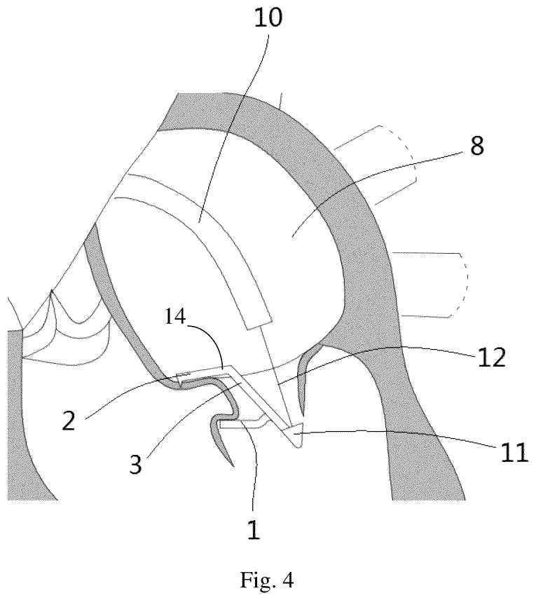

As shown in FIGS. 3-5, during the treatment procedure, the delivery apparatus 10 is inserted into the heart, until the tip 11 is positioned at the desired location with respect to the native leaflets. At this time, the proximal locking members 5 are unlocked and detached from the sheath 10, while the distal locking members 6 remain attached to the tip 11. The sheath 10 is then withdrawn by a suitable distance and separated from the tip 11, while the tip remains at the desired location due to the support of the wire 12. As a result, the treatment device 14, which is still attached to the tip 11 of delivery apparatus, is released from the sheath 10 and start to change its shape from the compressed state to the deployed state, as shown in FIG. 4. In the partially expanded state shown in FIG. 4, the anchoring structure 2 is expanded and rests over the native annulus tissue; the clamping structure 1 starts to push the native leaflets 13 upwardly. Then, the distal locking members 6 are unlocked and detached from the tip 11, and the tip is moved toward the left ventricle by a small distance, so that the treatment device 14 is completely released from the delivery apparatus 10. Consequently, the treatment device 14 changes to its fully deployed state, where the anchoring structure 2 is located in the left atrium abutting the annulus, and the clamping structure 1 is located in the left ventricle. In this state, the treatment device achieves treatment the native leaflets 13, e.g., adjusting the positions of the leaflets as described earlier. Subsequently, the delivery apparatus 10 including the tip 11 and the sheath 10 are withdrawn from the heart.

Any suitable attachment and locking/unlocking structures may be used inside the delivery apparatus to attach the proximal locking members 5 and distal locking members 6 to the sheath and the tip 11, respectively. Any suitable control mechanisms may be used to unlock them from the sheath 10 and the tip 11. This type of attachment and locking/unlocking structures and control mechanisms are known and used in various types of apparatus for performing minimally invasive surgery.

The above described native heart valve leaflet treatment system can treat the native leaflets of a human heart valve before the heart valve repair or replacement surgery (the repair or replacement surgery may be minimally-invasive surgery or open surgery). This treatment can ensure a more successful surgery, and reduce the complexity of the surgical procedure and the complexity of the surgical equipment needed, thereby reducing overall risk of the surgery.

By treating the native leaflets 13 before the heart valve repair or replace surgery according to embodiments of the present invention, the native leaflets 13 cab be treated without stopping the heart. The treatment process and result may be monitored in real time using an ultrasound imaging device, and corrections and further treatment may be performed if necessary based on the monitoring. This treatment solves the following problems of the currently technology: when open surgery is performed under cardiopulmonary bypass with the heart stopped, treatment result of native leaflets cannot be evaluated; when performing minimally invasive surgery, the native leaflets could not be treated before the heart valve repair or replacement surgery.

### Second Embodiment

This embodiment provides a method for treating native heart valve leaflets 13, which uses a minimally invasive procedure to fold or cut the native leaflets of the mitral valve leaflets or tricuspid valve before valve repair or replacement. The method is performed by a surgeon by manipulating the delivery apparatus, either directly or via a robotic system.

Using the native heart valve leaflet treatment system described in the first embodiment, the treatment method includes:

The treatment device 14 is folded and compressed, and loaded into the sheath 10 of the delivery apparatus. In one example, the clamping structure 1 is folded upwardly and located alongside the connecting bridges 3. At this time, the treatment device is in the compressed state for delivery.

The delivery apparatus 10 containing the treatment device is inserted along the inferior vena cava, through the right atrium 7 and the atrial septum, to enter the left atrium 8. The sheath of the delivery apparatus has adjustable bend. After reaching the left atrium 8, the distal portion of the delivery apparatus 10 forms an arc shape. A distal portion of the delivery apparatus including the tip 11 passes through the mitral valve to enter the left ventricle 9.

While maintaining the position of the tip 11, the sheath 10 is withdrawn by a suitable distance, to release the treatment device 14 from the sheath. The tip 11 is then moved by a suitable distance toward the left ventricle, to completely release the treatment device 14 from the delivery apparatus 10. Without the restraint of the delivery apparatus 10, the anchoring structure 2, the clamping structure 1 and the connecting bridges 3 (being made of a shape memory material) self-expand into the deployed state, i.e., taking their shape before they were compressed and loaded into the sheath.

In the deployed state, the piercing elements 4 pierce into the annulus tissue of the native valve to anchor the treatment device to the annulus. Meanwhile, the clamping structure 1 folds the native anterior leaflet by pushing it outwardly and upwardly toward the left atrium 8, and capturing a substantial portion of the leaflet in the space between the connecting bridges 3 and the clamping structure 1.

After the treatment device 14 is completely released and the clamping structure 1 captures and folds the native anterior leaflet, the entire delivery apparatus 10 is withdrawn from the body to complete the procedure.

### Additional Embodiments

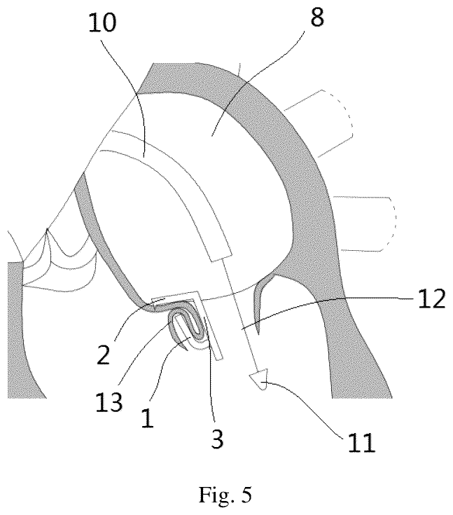

As described earlier, the treatment device 14 is C shaped in the top view, and extends around only a part of the annulus of the native heart valve. Preferably, the treatment device 14 is placed around the annulus at an angular position that overlaps with at least the native anterior leaflet to treat the anterior leaflet, as shown in FIGS. 4 and 5. The posterior leaflet, which is shorter, may be left untreated in this embodiment.

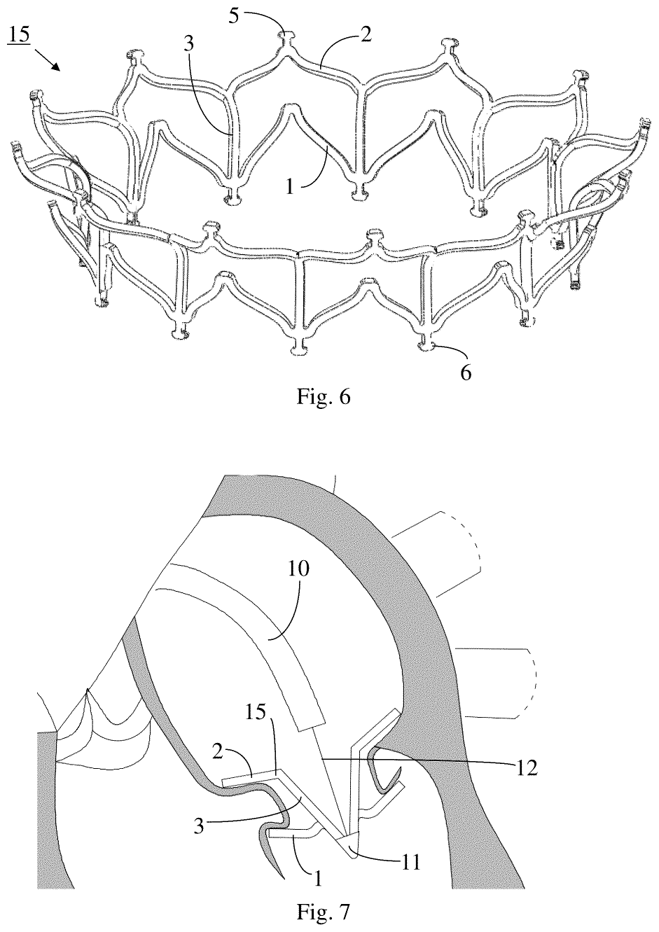

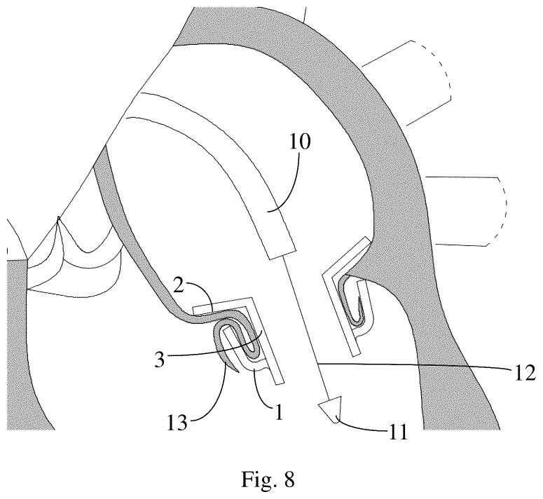

FIGS. 6-8 illustrate a native leaflet treatment device and system according to another embodiment of the present invention. As shown in FIG. 6, the native leaflet treatment device 15 of this embodiment is similar to the treatment device 14 shown in FIGS. 1, 1A, 2, 4 and 5, but is a closed ring structure (O shape) in the top view. Further, the piercing elements 4 are omitted, as the closed O shape can be securely placed around the entire native valve annulus even without the piercing elements 4. This treatment device 15 can treat (e.g. fold) both the native anterior and posterior leaflets, as shown in FIGS. 7 and 8. The related delivery system 10 and treatment method using the treatment device 15 are similar to those using the treatment device 14, as shown in FIGS. 7 and 8.

FIGS. 9-11 illustrate a native leaflet treatment device and system according to yet another embodiment of the present invention. As shown in FIG. 9, the native leaflet treatment device 16 of this embodiment is similar to the treatment device 15 shown in FIGS. 6-8, but the clamping structure 1 additionally includes a plurality of legs 17 joined to the clamping structure 1. In the illustrated embodiment, the legs 17 are joined at their proximal ends to the uppermost points of the undulating structure of the clamping structure 1, and bend downwardly. Further, instead of the distal locking members 6 joined to the clamping structure 1 as in the embodiments of FIGS. 1 and 6, the treatment device 16 of this embodiment has a plurality of distal locking members 18 located at the distal ends of the legs 17.

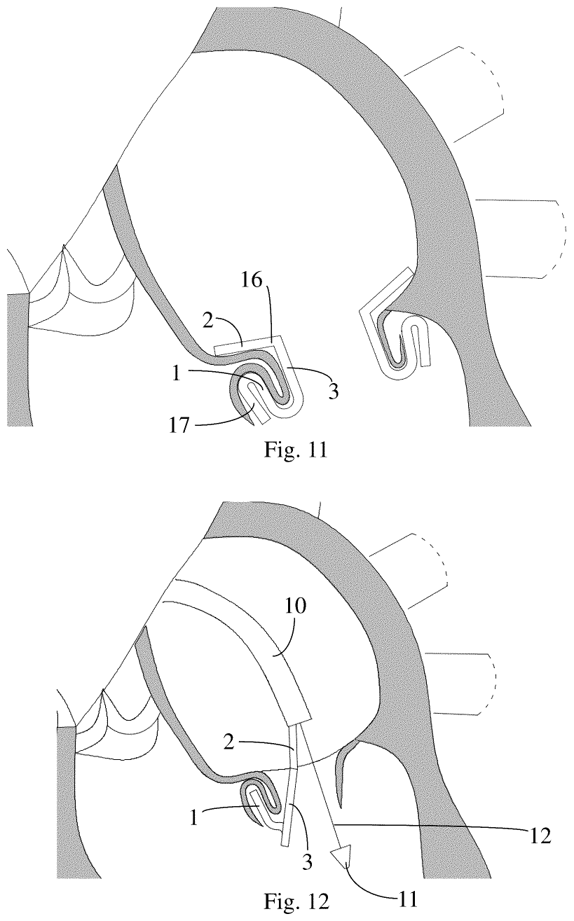

During deployment, as shown in FIG. 10, when the anchoring structure 2 has been released from the sheath and the distal locking members 18 are still retained in the tip 11, the clamping structure 1 is restrained by the legs 17 and does not fold fully upwards, but rather, assumes a near horizontal angle. Meanwhile, without the distal locking members 6, the connecting bridges 3 can expand more fully (compared with the embodiments of FIGS. 1 and 6). In this partially expanded state, the clamping structure 1 and legs 17 form a near-horizontal annular disk, which can better capture the native leaflets 13. Subsequently, the distal locking members 18 are released from the tip 11, and the treatment device 16 reaches the fully deployed state shown in FIG. 11. In the fully deployed state, the clamping structure 1 is folded upwardly, and the legs 17 bend downwardly, and the entirety or a substantial portion of the leaflets are captured in the space between the connecting bridges 3 and the clamping structure 1.

FIG. 12 illustrates an alternative deployment sequence for the treatment device 14 of the first embodiment of FIG. 1. In this alternative sequence, the clamping structure 1 and connecting bridges 3 are released from the sheath first and the distal locking members 6 are released from the tip 11 (or the distal locking members 6 may be omitted altogether), while the proximal end of the treatment device 14 including the proximal locking members 5 is still retained in the sheath 10, as shown in FIG. 12. In this partially expanded state, the clamping structure 1 and the lower portion of the connecting bridges 3 expand outwardly, folding the native leaflets 13 and capturing it in the space between the connecting bridges 3 and the clamping structure 1. Subsequently (not shown in the drawings), the proximal locking members 5 are unlocked and the treatment device 14 is completely released from the sheath 10. In the fully deployed stat, the anchoring structure 2 expands and folds outwardly to rest on the annulus tissue.

Similarly, the treatment devices 15 and 16 in the embodiments of FIGS. 6 and 9 may also be deployed in this alternative sequence, and detailed explanations are omitted here.

It should be noted that the native heart valve leaflet treatment devices according to various embodiments of the present invention are not replacement heart valves, and do not have prosthetic leaflets. Further, because their primary function is to capture and hold the native leaflets 13 (in particular, the anterior leaflet), the shape and size of the clamping structure 1 and connecting bridge 3 are designed to accommodate all or a substantial portion of the anterior native leaflets in the space between the clamping structure 1 and the connecting bridges 3, as illustrated in the side views in FIGS. 4, 5, 7, 8, 10 and 11. Thus, the lengths of the connecting bridges 3, i.e. the distance between their connection points with the anchoring structure 2 and corresponding connection points with the clamping structure 1, are preferably about one third to one half of the typical length of a anterior leaflet; the distance in the radial direction between the upper end of the clamping structure 1 and the connecting bridges 3 is preferably more than twice the typical thickness of a anterior leaflet.

It will be apparent to those skilled in the art that various modification and variations can be made in the heart valve native leaflet treatment method and apparatus of the present invention without departing from the spirit or scope of the invention. Thus, it is intended that the present invention cover modifications and variations that come within the scope of the appended claims and their equivalents.

What is claimed is:

1. A native leaflet treatment system for treating native leaflets of a native valve of a heart, comprising:
a delivery apparatus, including a sheath and a tip, configured to be inserted into the heart; and
a native leaflet treatment device, having a compressed state for delivery and a deployed state, wherein in the compressed state, the treatment device is compressed and stored inside the delivery apparatus, and in the deployed state, the treatment device is expanded,
wherein the treatment device includes a clamping structure, and in the deployed state, the clamping structure is configured to capture and retain at least a portion of the native leaflets.

2. The native leaflet treatment system of claim 1, wherein the treatment device further includes:
an anchoring structure; and
a plurality of connecting bridges connecting the clamping structure and the anchoring structure,
wherein the anchoring structure is connected at first ends of the connecting bridges, and wherein in the deployed state, the anchoring structure is configured to abut an annulus of the native valve.

3. The native leaflet treatment system of claim 2, wherein the treatment device further includes a plurality of piercing elements, connected to the anchoring structure and having sharp tips pointing toward the clamping structure, the sharp tips being configured to pierce into the annulus of the native valve in the deployed state.

4. The native leaflet treatment system of claim 2, wherein the clamping structure, the anchoring structure and the connecting bridges are formed of a shape memory material.

5. The native leaflet treatment system of claim 2, wherein the treatment device further includes a plurality of proximal locking members joined to the anchoring structure and configured to be detachably coupled to the sheath of the delivery apparatus when the treatment device is stored in the delivery apparatus.

6. The native leaflet treatment system of claim 5, wherein the treatment device further includes a plurality of distal locking members joined to the clamping structure and configured to be detachably coupled to the tip of the delivery apparatus when the treatment device is stored in the delivery apparatus.

7. The native leaflet treatment system of claim 6, wherein the plurality of proximal locking members are respectively joined to upper points of the anchoring structure, and the plurality of distal locking members are respectively joined to lower points of the clamping structure.

8. The native leaflet treatment system of claim 6, wherein the plurality of proximal locking members are respectively joined to upper points of the anchoring structure,
wherein the treatment device further includes a plurality of legs, wherein each leg is joined at its upper end to an upper point of the clamping structure and bends downwardly in the deployed state, and wherein the plurality of distal locking members are respectively joined to lower ends of the plurality of legs.

9. The native leaflet treatment system of claim 2, wherein each of the clamping structure and the anchoring structure is formed of connected struts, and
wherein in the deployed state, each of the clamping structure and the anchoring structure forms an undulating structure, each connecting bridge is connected to a lower point of the undulating structure of the anchoring structure and a lower point of the undulating structure of the clamping structure, and both the clamping structure and the anchoring structure extend upwardly and outwardly from the respective lower points where they are joined to the connecting bridges.

10. The native leaflet treatment system of claim 2, wherein in the deployed state, the anchoring structure forms an angle between 0 and 75 degrees with respect to a horizontal plane of the treatment device.

11. The native leaflet treatment system of claim 1, wherein the treatment device is formed of a plurality of repeating units connected to each other and arranged in an angular direction around an longitudinal axis of the treatment device.

12. The native leaflet treatment system of claim 11, wherein in a top view in the deployed state, the treatment device has an open shape forming an incomplete ring around the longitudinal axis.

13. The native leaflet treatment system of claim 11, wherein in a top view in the deployed state, the treatment device has a closed shape forming a complete ring around the longitudinal axis.

14. The native leaflet treatment system of claim 1, wherein the delivery apparatus further includes a wire which passes through the sheath and is connected at its distal end to the tip of the delivery apparatus.

15. A native leaflet treatment method for treating the native leaflets using a native leaflet treatment system, the treatment system including a delivery apparatus and a native leaflet treatment device stored inside the delivery apparatus in a compressed state, the native leaflet treatment device including a clamping structure, and the native leaflet treatment device being configured to self-expand to a deployed state upon release from the delivery apparatus, the method comprising:
inserting the delivery apparatus with the treatment device stored therein into an atrium of the heart and through the native valve;
releasing the treatment device from the delivery apparatus, wherein the treatment device self-expands to the deployed state wherein the clamping structure captures and retains at least a portion of the native leaflets; and
withdrawing the delivery apparatus from the heart.

16. The native leaflet treatment method of claim 15, wherein the delivery apparatus including a sheath and a tip located a s distal end of the sheath, and wherein the releasing step includes:
releasing a proximal portion of the treatment device from the sheath while retaining a distal end of the treatment device in the tip, wherein the released proximal portion of the treatment device self-expands to abut an annulus of the native valve; and
thereafter, releasing the distal end of the treatment device from the tip.

17. The native leaflet treatment method of claim 15, wherein the delivery apparatus including a sheath and a tip located a s distal end of the sheath, and wherein the releasing step includes:
releasing a distal portion of the treatment device including the clamping structure from the tip and the sheath while retaining a proximal end of the treatment device in the sheath, wherein the clamping structure self-expands to capture and retain at least a portion of the native leaflets; and
thereafter, releasing the proximal end of the treatment device from the sheath, wherein the treatment device self-expands further to abut an annulus of the native valve.

18. The native leaflet treatment method of claim 15, wherein the treatment device in the deployed state is anchored to an annulus of the native valve by a plurality of piercing elements.

19. A native leaflet treatment method of treating native leaflets of a native valve of a heart, comprising delivering a treatment device into the heart transcatheterly, wherein the treatment device self-expands to capture and retain the native leaflets, before repairing or replacing the native valve.

20. The method of claim 19, wherein the atrium is the left atrium and the native valve is a mitral valve.
